(19) United States
(12) Patent Application Publication
(10) Pub. No.: US 2026/0168893 A1
(43) Pub. Date: Jun. 18, 2026

(54) SYSTEMS AND METHODS FOR DETECTING AND CHARACTERIZING CONTAMINATION IN GAS SAMPLING SYSTEMS

(71) Applicant: Schlumberger Technology Corporation, Sugar Land, TX (US)

(72) Inventors: Elena Borisova, Paris (FR); Jean-Pierre Bailly, Clamart (FR)

(21) Appl. No.: 19/293,278

(22) Filed: Aug. 7, 2025

(30) Foreign Application Priority Data
Dec. 12, 2024 (EP) 24307096.8

(51) Int. Cl. G01N 1/24; E21B 21/06; E21B 49/08; G01N 33/28

(52) U.S. Cl. CPC G01N 1/24; E21B 21/067; E21B 49/086; G01N 33/2823

(57) ABSTRACT

Provided herein are gas analysis systems comprising a sampling circuit and a gas analyzer operable to measure a gas flowing through the sampling circuit. A purge fluid may be routed through the sampling circuit and measured by the gas analyzer to detect contamination in the sampling circuit. One or more remedial actions may be taken, by an operator and/or a controller, in response to detected contamination in the sampling circuit to improve the reliability, accuracy, and/or longevity of the gas analysis system.

### CROSS-REFERENCE TO RELATED APPLICATIONS

This application claims priority to and benefit of EP Patent Application No. 24307096.8, filed on December 12, 2024, which is incorporated by reference herein in its entirety.

### BACKGROUND

The field of disclosure relates generally to systems and methods for analyzing gas contained in a drilling fluid exiting a wellbore.

During the drilling of a petroleum or gas well, gas drilling compounds (e.g., hydrocarbons, carbon dioxide, hydrogen sulfide, nitrogen) contained in the drilling fluid (or “drilling mud”) emerging from the well can be analyzed to enable reconstructing the geological succession of the crossed layers of the geological formation during drilling and determining the opportunities of exploiting fluids contained in the geological formation. For example, gas chromatography is a widely used analysis technique in the petroleum industry due to its various applications – from the exploration of crude oil and refining of finished products to research on new petrochemicals.

The analysis of the gas drilling compounds in the drilling mud (also referred to as “mud logging”) can be performed continuously, in two main phases. First, the drilling mud is sampled, and the sampled drilling mud is “degassed” to extract the gas drilling compounds from the mud. Second, the extracted gases are transferred through a sampling circuit, towards an analyzer (e.g., a gas chromatograph) that can identify and, in some instances, quantify the extracted gases.

An ongoing challenge in mud logging is improving the accuracy and reliability of the analyzer. Occasionally, or over time, the sampling circuit can become contaminated, which creates the risk of contaminating the extracted gases, and ultimately may lead to inaccurate or unreliable results of the analyzer. For example, the certain compounds in the extracted gases may have the propensity to adsorb on internal surfaces of pipes or conduits of the sampling circuit, and may be released back into the gas sample thereby negatively influencing the gas analysis. At present, the detection of contamination in the sample circuit is not systematic. Instead, the operator’s vigilance and skills must be relied upon to predict or detect contamination and, even then, the contamination is often detected too late, when the data are already in the process of geological interpretation.

Accordingly, systems and methods that enable more accurate and reliable analysis of extracted gas samples are needed. In particular, there is a need in the industry for systems and methods that can detect the existence of contamination in extracted gas samples, thereby enabling remedial action to be taken to remove the source of contamination and/or enabling adjustment of the analysis equipment to compensate for any contamination in the gas samples.

### SUMMARY

In one aspect, provided herein is a gas analysis system, the system comprising a sampling circuit and a gas analyzer connected to the sampling circuit. The sampling circuit may comprise a conduit connectable to a gas supply and to a purge fluid supply. The conduit when connected to the gas supply may receive a gas sample. The conduit when connected to the purge fluid supply may receive a purge fluid. The sampling circuit may also comprise at least one valve positionable to enable connection between the conduit and the gas supply and to enable connection between the conduit and the purge fluid supply. The at least one valve may be positioned to connect the conduit and the gas supply during a sample analysis operation. The at least one valve may be positioned to connect the conduit and the purge fluid supply during a contaminant analysis operation. The gas analyzer may be operable to obtain measurement data indicative of gas content in the gas sample during the sample analysis operation and measurement data indicative of gas content in the purge fluid during the contaminant analysis operation.

In another aspect, provided herein is a well site installation for analyzing drilling fluid exiting a wellbore, the installation comprising a pipe drawing the drilling fluid from the wellbore, a sampling circuit in fluid communication with the pipe, and a gas analyzer connected to the sampling circuit. The sampling circuit may comprise an extractor operable to separate gas contained in the drilling fluid, a conduit, and at least one valve. The conduit may be connectable to the extractor for receiving a sample of the gas. The conduit may be connectable to a purge fluid supply for receiving a purge fluid. The at least one valve may be positionable to enable connection between the conduit and the extractor and to enable connection between the conduit and the purge fluid supply. The at least one valve may be positioned to connect the conduit and the extractor during a sample analysis operation. The at least one valve may be positioned to connect the conduit and the purge fluid supply during the contaminant analysis operation. The gas analyzer may be operable to obtain measurement data indicative of gas content in the sample of the gas during the sample analysis operation and measurement data indicative of gas content in the purge fluid during the contaminant analysis operation.

In another aspect, provided herein is a gas analysis system comprising a sampling circuit, a gas analyzer connected to the sampling circuit, and a computing device electronically connected to the gas analyzer. The sampling circuit may be connectable to a gas supply for receiving a gas sample during a sample analysis operation. The gas analyzer may be operable to obtain measurement data indicative of gas content in the gas sample during the sample analysis operation and to obtain measurement data indicative of contaminant in the sampling circuit when the sample analysis operation is not being performed. The computing device may include a processor and a memory storing instructions that, when executed by the processor, may cause the processor to detect the contaminant in the sampling circuit based on the measurement data obtained by the gas analyzer when the sample analysis operation is not being performed. The instructions, when executed, may also cause the processor to adjust a measurement setting of the gas analyzer in response to the detected contaminant.

In another aspect, provided herein is a well site installation for analyzing drilling fluid exiting a wellbore, the installation comprising a pipe drawing the drilling fluid from the wellbore, a sampling circuit in fluid communication with the pipe, a gas analyzer connected to the sampling circuit, and a computing device electronically connected to the gas analyzer. The sampling circuit may be operable to separate gas contained in the drilling fluid and receive a sample of the gas during a sample analysis operation. The gas analyzer may be operable to obtain measurement data indicative of gas content in the gas sample during the sample analysis operation and to obtain measurement data indicative of contaminant in the sampling circuit when the sample analysis operation is not being performed. The computing device may include a processor and a memory storing instructions that, when executed by the processor, cause the processor to detect the contaminant in the sampling circuit based on the measurement data obtained by the gas analyzer when the sample analysis operation is not being performed. The instructions, when executed, may also cause the processor to adjust a measurement setting of the gas analyzer in response to the detected contaminant.

In another aspect, provided herein is a method of analyzing gas contained in a working fluid, the method comprising: sampling the gas via a sampling circuit connected to a gas analyzer; measuring the gas using the gas analyzer to detect one or more gas compounds in the gas; intermittently ceasing the sampling of the gas and, when the sampling is ceased, purging the sampling circuit with a purge fluid; and measuring the purge fluid using the gas analyzer to detect one or more contaminants in the sampling circuit.

The following description and the appended figures set forth certain features for purposes of illustration.

### BRIEF DESCRIPTION OF DRAWINGS

So that the manner where the above recited features may be understood in detail, a more particular description, briefly summarized above, may be had by reference to example aspects, some of which are illustrated in the appended drawings.

FIG. 1 is a schematic of a well site installation including a system for analyzing drilling fluid exiting the wellbore, according to an embodiment of the present disclosure.

FIG. 2 is a schematic of a portion of a sampling circuit usable with the well site installation of FIG. 1, according to an embodiment of the present disclosure.

FIG. 3 is a schematic of a computing device of the sampling circuit of FIG. 2, according to an embodiment of the present disclosure.

FIG. 4 is an example process flow diagram according to embodiments of the present disclosure.

FIG. 5 is an example chromatograph depicting a rate of decontamination that may be monitored during a contaminant analysis operation of the present disclosure.

### DETAILED DESCRIPTION

One or more specific embodiments of the present disclosure will be described herein. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers’ specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure generally relates to systems and methods for analyzing a gas contained in a working fluid. In some embodiments, the systems and methods can be used to analyze gas contained in a drilling fluid exiting a wellbore of a well site installation. The gas is analyzed during a sample analysis operation using a gas analyzer (e.g., a gas chromatography system) that is operable to detect gas compounds in the gas. The information related to detected gas compounds can be used to make various determinations regarding the working fluid. For example, in well site installation applications, the gas compounds (e.g., hydrocarbons) that are present in the gas contained in the drilling fluid can provide insight into the geological formation being drilled and the drilling opportunities that are available (e.g., oil production potential), as well as help navigate the drilling process (e.g., by enabling the identification of pay zones or “sweet spots”). Although embodiments of the present disclosure are described with respect to well site installation applications, the present disclosure is not limited to such applications. Aspects of the present disclosure as they relate to analyzing gas contained in a working fluid can be used in any suitable application (e.g., in an industrial or manufacturing process where it is desirable to monitor the composition of a gas contained in a working fluid).

In accordance with the present disclosure, the embodiments described herein include a sampling circuit that is connected to the gas analyzer and operates to sample the gas contained in the working fluid. The sampling circuit may occasionally or over time become contaminated (e.g., via adsorption of gas compounds within conduits or working components of the sampling circuit), which negatively affects the ability of the gas analyzer to provide accurate and reliable measurement results regarding the sampled gas. Accordingly, the gas analyzer is operable to intermittently measure contamination within the sampling circuit, and the detection of such contamination can be used to take one or more remedial actions for removing the contamination and/or adjusting the gas analyzer to compensate for the contamination and improve accuracy and reliability of its measurements. To measure contamination in the sampling circuit, a purge fluid (e.g., air or an inert gas) can be routed through the sampling circuit towards the gas analyzer when the gas contained in the working fluid is not being sampled. Such “contaminant analysis operations” can advantageously be performed during standard downtime procedures in the field in which the working fluid is not available for sampling to minimize or eliminate any production inefficiencies that may otherwise be caused by routing the purge fluid through the sampling circuit. For example, in well site installation applications, a contaminant analysis operation can be performed when another segment of a drill pipe is being installed.

In some embodiments, the sampling circuit includes at least one valve (e.g., a switching or three-way valve) that is positionable to provide connection between the sampling circuit and a supply of the gas for sampling during a sample analysis operation, and to provide connection between the sampling circuit and a supply of the purge fluid during a contaminant analysis operation. The valve thereby isolates the sampling circuit from the gas supply when the purge fluid is being routed through the sampling circuit, and isolates the sampling circuit from the purge fluid supply when the gas sample is being routed through the sampling circuit, to avoid cross-contamination of the gas sample and purge fluid that can otherwise cause unreliable and inaccurate measurement. This enables switching between the sample analysis and contaminant analysis operations efficiently at low cost and with minimum complexity. Additionally, in some embodiments, the sampling circuit can be connectable with the purge fluid supply at multiple locations along the sampling circuit. For example, the sampling circuit can be connectable with the purge fluid supply both upstream and downstream from a pump of the sampling circuit and/or both upstream and downstream from a filter of the sampling circuit. In this way, detection of contamination in the sampling circuit can be made with greater flexibility and discrete contamination zones within the sampling circuit can be identified.

In some embodiments, a computing device is electronically connected to the gas analyzer and includes a processor and a memory storing instructions executable by the processor to perform various functions for controlling the gas analyzer and/or storing measurement data from the gas analyzer. The computing device can store in the memory predefined thresholds for various contaminants (e.g., various hydrocarbons) that, when exceeded in measurements obtained by the gas analyzer during contaminant analysis, signal to the processor that contaminant is present in the sampling circuit. In response to the detected contaminant in the sampling circuit, the processor can alert an operator of the contamination and/or adjust a measurement setting of the gas. The adjusted measurement setting can be used to obtain more comprehensive measurement data of contaminants in the sampling circuit during the contaminant analysis and/or to compensate for the contamination during subsequent sample analysis. Additionally, or alternatively, the processor can store the measurement data indicative of contamination in the sampling circuit in the memory, which can be used to detect future contamination events in the sampling circuit and/or used for more accurate and reliable identification of contaminants in the sampling circuit. Additionally, or alternatively, the processor can continue to monitor a level of contamination during the contaminant analysis to identify and/or assess a rate of decontamination of the sampling circuit as the purge fluid is routed therethrough. In this regard, various remedial and/or responsive actions can be taken automatically and/or in real-time by the computing device when a contamination event in the sampling circuit is detected.

Advantageously, the contaminant analysis operation of the present disclosure enables the obtention of “fingerprint” measurement data that can be stored in the memory of the computing device and provides information on the contamination of the sampling circuit. In some embodiments, the gas analyzer is a gas chromatography system and fingerprint chromatograms of the sampling circuit contamination can be obtained and stored in the memory of the computing device. In these embodiments, the fingerprint chromatograms contain information on certain types of contamination of that have historically been detected in the sampling circuit, with the retention times of the contaminant(s) enabling their identification and the decrease of the amplitude over time (e.g., over multiple cycles performed during the contaminant analysis) indicating the dynamics of decontamination which can enable even more precise identification of the type of contaminants. The fingerprint chromatograms (or other measurement data) can provide numerous benefits including, but not limited to including: i) archiving chromatographic data in the memory of the computing device together with other parameters of the sampling circuit (e.g., temperature and/or pressure) for building a comprehensive knowledge database of sampling circuit; ii) enabling automatic comparison being chromatographic data and historical contamination data in the knowledge database and real-time identification of contaminants and suggestion of remedial actions; iii) transmitting chromatographic data to remote subject matter experts to identify contaminants and receive appropriate guidelines; iv) enabling use of peak location and shape for automatic identification of target components whose measurement is affected by contaminants; v) enabling use of peak location and shape as input for a peak processing algorithm to make appropriate corrections for interpretation and analysis of the measurement data obtained during sample analysis; vi) enabling use of peak location and shape can be used as an input for correction of total gas measurement data; vii) analysis of decontamination dynamics can enable suggestions for remedial actions such as switching to another pump or changing components in the sampling circuit; and/or viii) enabling wider access to and leverage of “contamination fingerprint” data industry-wide for additional real-time information on the underlying (e.g., drilling) process.

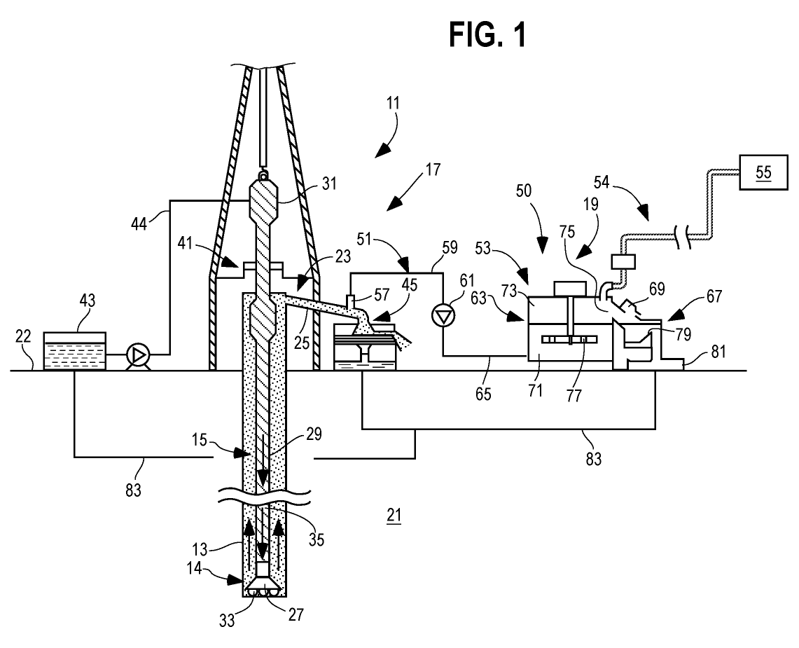

Referring now to the drawings, FIG. 1 is a schematic drawing of an installation 11 according to an embodiment of the present disclosure. The installation 11 in this example is a drilling installation of a well site for producing fluid, e.g., hydrocarbons, such as an oil well site. The installation 11 includes a borehole 13 delimiting a cavity 14 formed in a geological formation 21. The cavity is formed by a rotary drilling tool 15, and drilling pipes are placed in the cavity 14. The installation also includes a surface installation 17, and a system 19 for analyzing a drilling fluid. At a surface 22 of the geological formation 21, a well head 23 having a discharge pipe 25 closes the borehole 13.

The drilling tool 15 includes a drilling head 27, a drill string 29 and a liquid injection head 31. The drilling head 27 includes a drill bit 33 for drilling through the rocks of the geological formation 21. The drill bit 33 is mounted on the lower portion of the drill string 29 and is positioned in the bottom of the drilling pipe in the borehole 13.

The drill string 29 includes a set of hollow drilling pipes that delimit an internal space 35. The internal space 35 enables channeling a drilling fluid from the surface 22 to the drilling head 27. To this end, the liquid injection head 31 is fastened (e.g., screwed onto) the upper portion of the drill string 29. The drilling fluid is a drilling mud, such as a water-based, oil-based, or synthetic-based drilling mud. After having flown downwards to the drill bit 33, the drilling fluid flows upwards in the space created between the drill string 35 and a wall of the borehole 13 and is discharged in a discharge pipe 25 of the surface installation.

The surface installation 17 includes a support 41 for supporting the drilling tool 15 and driving it in rotation, an injector 43 for injecting the drilling liquid connected to an injection line 44 for transporting drilling fluid, and a shale shaker 45. The injector 43 is hydraulically connected to the injection head 31 via an injection pipe 44 to introduce and circulate the drilling fluid in the inner space 35 of the drill string 29. The shale shaker 45 collects the drilling fluid charged with drilling residues, known as cuttings, the drilling fluid flowing out from the discharge pipe 25. The shale shaker 45 equipped with sieves allows the separation of the solid drilling residues from the drilling mud.

The analysis system 19 includes a sampling circuit 50 that receives a sample of the drilling mud from the discharge pipe 25 and extracts a gas sample from the drilling mud, and an analyzer or analysis device 55 that obtains measurement data indicative of gas content in the gas sample. The sampling circuit 50 includes a mud sampler 51 that draws a sample of the mud from the discharge pipe 25, and a gas extractor 53 connected to the mud sampler 51. The gas extractor 53 operates to “degas” the sample of the mud drawn from the mud sampler 51 and extract gas drilling compounds from the mud. The sampling circuit 50 also includes a line or conduit 54 allowing the transport of the extracted gases from the extractor 53 to the analyzer 55.

The mud sampler 51 includes a liquid sampling device 57, connected to the discharge pipe 25, a duct 59, and a pump 61 (e.g., a peristaltic pump) with an adjustable flow rate. The duct 59 connects the liquid sampling device 57 to the gas extractor 53. The duct 59 may be provided with a mud heater that raises the temperature of the mud to values between 5°C to 150°C, for example, between 50°C to 100°C.

The gas extractor 53 includes an enclosure 63, a pipe 65 that supplies mud to the enclosure 63, a pipe 67 that discharges the mud from the enclosure 63, and an inlet 69 that introduces gas (e.g., air) into the enclosure 63. The inlet 69 is tapped in this embodiment into the discharging pipe 67. The enclosure 63 is provided with an agitator 77 mounted in a projecting manner in the enclosure 63 and rotatably driven by a motor mounted on the enclosure 63. The agitator 77 includes an agitating moving body immersed in the mud. The agitator 77 is operable to vigorously stir the mud to extract gases therefrom. In some embodiments, the enclosure 63 may be thermally connected to a heater for raising a temperature of the mud, in addition to or in the alternative to the mud heater of the duct 59 described above, to further enable extracting gases from the mud.

The pipe 65 that supplies mud to the enclosure 63 extends between an outlet of the pump 61 and an inlet opening provided in a lower portion 71 of the enclosure 63. The discharge pipe 67 extends between an overflow passage 75 provided in the enclosure 63, and a retaining pit 81 that receives the mud discharged from the extractor 53. The mud collected in the retaining pit 81 is recycled to the injector 43 by way of a pipe 83 for recirculating mud. The discharge conduit 75 is advantageously bent to form a siphon 79 opening out and facing the retaining pit 81 above the level of liquid contained in the pit 81.

The enclosure 63 has an inner volume that defines the lower portion 71, in which circulates the drilling fluid stemming from the supply pipe 65, and an upper portion 73 defining a gas head or clearance space above the drilling fluid. The volumes of the lower portion 71 and the upper portion 73 may be maintained substantially constant in operation.

The mud introduced into the lower portion 71 of the enclosure 63 via the supply pipe 65 is discharged by overflowing into the discharge pipe 67 through the overflow passage 75. In addition, some of the mud discharged dwells temporarily in the siphon 79 of the discharge pipe 67, which prevents gas from entering the upper portion 73 of the enclosure 63 by way of the lower end of the discharge pipe 67. The inlet 69 thereby operates as the only means for introducing gas into the enclosure 63 in this example.

The inlet 69 includes an air intake mounted on the upstream portion of the discharge pipe 67. This air intake opens into an injection passage in the discharge pipe 67. In the example illustrated, the gas is constituted by the air surrounding the installation, at atmospheric pressure, so that the pressure in the enclosure 63 is kept substantially constant at the atmospheric pressure when the injection passage is open. In some embodiments, the gas introduced via the inlet 69 includes air and/or nitrogen.

The gas extractor disclosed in the above-mentioned embodiment is exemplary may be replaced by any other type of extractor (or gas trap or degasser) operable to extract gas contained in a drilling fluid.

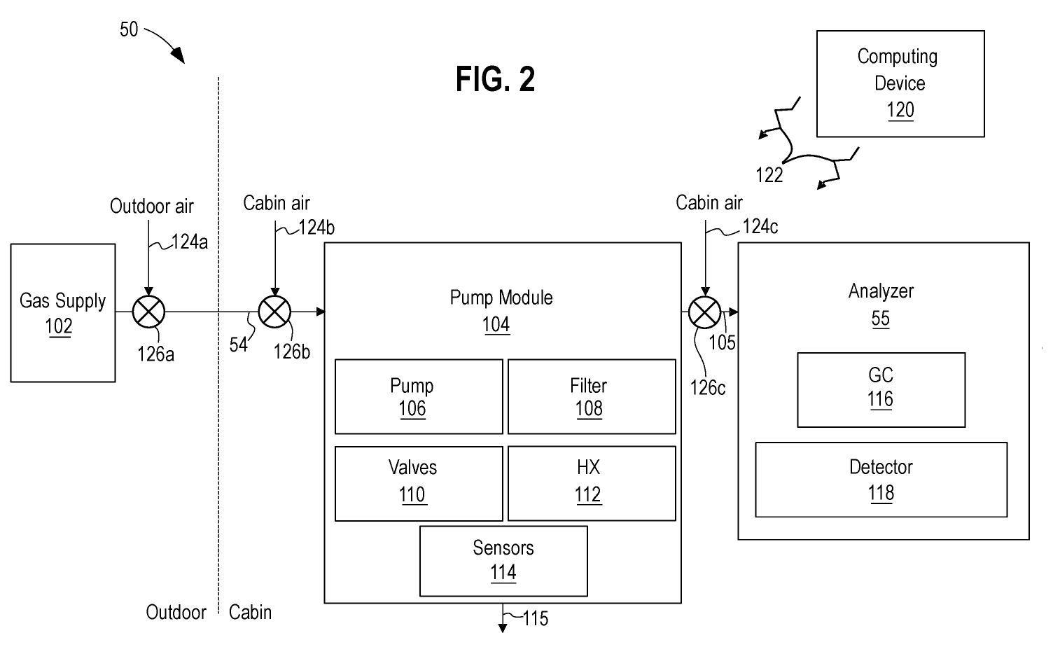

The transport line 54 connects the gas extractor 53 and the analyzer 55, which is operable to detect and quantify gas compounds or gas fractions contained in the extracted gases received from the gas extractor 53 via the line 54. The line 54 can be in any suitable material used for tubing realization and is advantageously made on the basis of a metal or polymer material, such as polyethylene and/or polytetrafluoroethylene (PTFE) for example. As discussed below with reference to FIG. 2, the sampling circuit 50 is equipped with a pump module 104 that includes a fluid motive device 106 (e.g., a pump) operable to convey the extracted gases from the gas extractor 53 through the line 54 towards the analyzer 55. The line 54 is tapped into the upper portion 73 of the enclosure 63 of the gas extractor 53 for receiving samples of the extracted gases in the enclosure 63.

Referring now to FIG. 2, a schematic of a portion of the sampling circuit 50 of FIG. 1 including the analyzer 55 and the line 54 extending from the gas extractor 53 to the analyzer 55 is shown. The gas extractor 53 is represented in this illustration at gas supply 102. Although the gas supply 102 may be the gas extractor 53 in this embodiment, the gas supply 102 can include any supply of gases for analysis using the analyzer 55. Accordingly, as the description proceeds, the gas extractor 53 may be referred to more broadly as the gas supply 102.

The analyzer 55 can include any suitable configuration to enable the analyzer to perform the functions described herein. In some examples, the analyzer 55 includes similar features and components as the analysis devices described in U.S. Patent No. 10,655,464, issued May 19, 2020, and/or the analysis devices described in EP Patent No. 3772648, published March 27, 2024, the entire disclosures of which are hereby incorporated by reference.

In various embodiments of the present disclosure, the analyzer 55 includes a gas chromatography system having a gas separation module 116 (e.g., a gas chromatograph or GC) separating the different gas compounds or gas fractions contained in the extracted gases and an analyzing instrumentation 118 (also referred to as a detector) downstream from the gas chromatograph detecting and quantifying the gas compounds or gas fractions in the extracted gases.

The detector 118 may include, for example, any one or more of the following: FID (flame ionization detector), TCD (thermal conductivity detector), Fourier Transform InfraRed (FTIR) spectrometer, Laser Photo Acoustic Spectrometer (LPAS), Pulse discharge detector (PDD), Photoionization Detector (PID), Electron Capture Detector (ECD) or mass spectrometer (MS). The detector 118 may vary depending on the extracted gases of interest. In well site installation applications, for example, the gas compounds of interest for analysis include hydrocarbons, such as aliphatic or aromatic C₁-C₁₀ hydrocarbons, alkanes, and/or alkenes, hydrogen sulfide, etc. In some examples, the detector 118 can be used without the gas chromatograph 116 or any other gas separation module.

The detector 118 can obtain measurement data that identifies and quantifies one or more gas compounds or gas fractions contained in the extracted gases. Additionally, or alternatively, the detector 118 can obtain measurement data that detects and quantifies a total content of the extracted gas (“total gas”).

In the illustrated example, the pump module 104 and the analyzer 55 are each located in a cabin and located remotely from the well site and the installation 11. The transport line 54 extends between and connects the gas supply 102 located outdoors at or near the well site and the pump module 104 and the analyzer 55 located in the cabin. The cabin can be located out of the ATEX 1 zone (e.g., several hectometers from the well site and the installation). For example, the transport line 54 may be longer than 10 meters (m), such as between 50 m to 500 m in length.

Alternatively, part or the totality of the analyzer 55 may be situated on the well site, or in the vicinity of the well site, for instance in the ATEX 1 zone. In some embodiments, the pump module 104, the gas separation module 116 (e.g., a gas chromatograph or GC), and/or the detector 118 of the analyzer 55 are situated at the well site, and a computing device 120 of the sampling circuit 50 may be situated at the well site or remotely from the well site. In such embodiments, the transport line 54 may be shorter than 50 m, or shorter than 10 m. Part or the totality of the analyzer 55 may additionally or alternatively be situated in a remote location away from the well site, for instance in the cabin situated out of the ATEX 1 zone. In some embodiments, the analyzer 55 includes a plurality of modules, wherein one of the modules may be situated at the well site, and at least another of the modules may be situated remotely from the well site, for instance in the cabin or at a supervision center for supervising a plurality of well sites. In some embodiments, the totality of the analyzer 55 is located remotely from the well site in a non-explosive area. In this regard, the line 54 can have any suitable length for transporting the extracted gases from the extractor 53 to the analyzer 55, and the length of the line 54 can vary depending on the location of the analyzer 55. In various embodiments, for example, the line 54 may have a length between 1 m to 500 m.

The pump module 104 may be located upstream from the analyzer 55 and may include the fluid motive device 106 (e.g., a pump) that operates to convey the gases from the gas supply 102 towards the analyzer 55. The pump module 104 may also include one or more additional components that condition the gases upstream from the analyzer 55 and/or control one or more parameters of the gases 55 prior to separation in the gas chromatograph 116 and/or analysis in the detector 118. For example, the pump module 104 may include one or more filters 108 (e.g., syringe filters) that filter dust, liquids, and/or particles from the gases upstream from the analyzer 55. Additionally, or alternatively, the pump module 104 may include one or more valves 110 (e.g., proportioning valves) that control a pressure and/or flow of the gases upstream from the analyzer 55. Additionally, or alternatively, the pump module 104 may include one or more thermal control modules or heat exchangers 112 (e.g., a Peltier module) that control a temperature of the gases upstream from the analyzer 55.

The pump module 104 may monitor the parameters (e.g., flow, temperature, and/or pressure) of the gases upstream from the analyzer 55 via one or more sensors 114. The monitored parameters via the sensors 114 may be used as feedback to control the pump 106, the valve(s) 110, the thermal control module(s) 112, and/or another one or more components of the pump module 104 using a feedback control loop. The pump module 104 may include a dedicated controller, and/or may be in communication with the computing device 120 that operates to control the pump module 104. The computing device 120 may include a central controller or may be a distributed network of controllers operable to communicate with and/or monitor components of the sampling circuit 50, such as the pump module 104.

In some examples, the pump module 104 may include a vent line 115 venting gases from the transport line 54 and pump module 124, and thereby circumventing the analyzer 55. The vent line 115 may include one or more of the valves 110 for controlling gas flow therethrough. The vent line 115 may also include one or more of the sensors 114 (e.g., a flow sensor) for monitoring parameters of the gas flowing through the vent line 115, which may be used to control the valve(s) 110 on the vent line 115. In some examples, the vent line 115 may include one or more sampling ports for extracting a sample of the gases in the vent line 115. Additionally, or alternatively, the pump module 104 may include sampling ports at any desired location.

The components of the pump module 104 can be arranged in any suitable configuration. The filter(s) 108, the valve(s) 110, the thermal control module(s) 112, the sensor(s) 114, and/or the vent line 115 can be located upstream or downstream from the pump 106, in any combination. The pump module 104 can be configured to control output of the gases to the analyzer 55 at a desired or specified flow, temperature and/or pressure. The parameters (e.g., flow, temperature and/or pressure) of the gases exiting the pump module 104 upstream from the analyzer 55 may vary depending on, for example, the type of gases being analyzed, the operating conditions of the gas supply 102, the type of analyzer 55, specifications of the analyzer 55, among other considerations.

Still referring to FIG. 2, the sampling circuit 50 preferably includes one or more purge lines 124 that introduce a purge fluid into the sampling circuit 50 via a purge fluid supply. The purge fluid can be intermittently or periodically introduced into the sampling circuit 50, and allows the analyzer 55 to measure contamination in the sampling circuit 50 while flow of the gases from the gas supply 102 is ceased. For example, the purge fluid can include a fluid with a known or calibrated composition, and any anomalies measured by the analyzer 55 while the purge fluid is routed through the sampling circuit 50 can be used to detect or signal contamination within the sampling circuit 50. The sampling circuit 50 can occasionally or over time become contaminated within various components via adsorption of gas compounds. For example, gas compounds may adsorb on internal surfaces within the transport line 54, the pump module 104, and/or in a conduit 105 extending between the pump module 104 and the analyzer 55. The gas compounds can subsequently desorb into the gas stream during analysis, which can negatively affect the ability of the analyzer 55 to provide accurate and reliable measurement results regarding the sampled gas. Accordingly, any contamination measured in the purge fluid by the analyzer 55 can be used to correct or re-calibrate the analyzer 55 to compensate for the contamination and/or take another remedial action including, but not limited to, those described herein.

The purge fluid introduced via the lines 124 may include any fluid that is measurable using the analyzer 55 and can be used to detect anomalies indicative of contamination (e.g., hydrocarbon contamination). Suitable purge fluids can include air and/or inert gas (e.g., nitrogen, argon, helium, and the like). In some embodiments, the purge fluid can be routed from the outdoors (e.g., via a first purge line 124a) and/or the purge fluid can be routed from the cabin (e.g., via a second purge line 124b or a third purge line 124c).

Any number of purge lines 124 can be included in the sampling circuit 50, such as one or more than one (e.g., two, three, four, five, or more than five) purge line 124. The purge lines 124 can also be strategically located in the sampling circuit 50 to include or bypass components of the sampling circuit 50 to selectively provide more comprehensive contamination detection or more acute contamination detection within discrete zones of the sampling circuit 50. In the illustrated example of FIG. 2, three purge lines 124 are included and labeled 124a-c.

The first purge line 124a is located on the transport line 54, upstream from the pump module 104 and proximate to the outlet of the gas supply 102 where the extracted gases are received in the transport line 54. Purge fluid (e.g., outside air) introduced via the first purge line 124a may provide a comprehensive contamination analysis when measured by the analyzer 55, since it may travel through a substantial entirety of the transport line 54 in addition to the pump module 104 and the conduit 105.

The second purge line 124b is located on the transport line 54, upstream from and in close proximity to the inlet of the pump module 104. Purge fluid (e.g., cabin air) introduced via the second purge line 124b may provide a relatively more acute contamination analysis when measured by the analyzer 55, since it may substantially bypass the transport line 54. Accordingly, the purge fluid may be introduced via the second purge line 124b for discrete contamination analysis within the pump module 104.

The third purge line 124c is located on the conduit 105 downstream from the pump module 104 and upstream from the analyzer 55. Purge fluid (e.g., cabin air) introduced via the third purge line 124c may provide the most acute contamination analysis of the purge lines 124 when measured by the analyzer 55, since it may substantially bypass both the transport line 54 and the pump module 104. Accordingly, the purge fluid may be introduced via the third purge line 124c for discrete contamination analysis of components downstream from the pump module 104. Additionally, or alternatively, the third purge line 124c can be used to introduce the purge fluid for calibrating the analyzer 55 prior to introducing the purge fluid at a more upstream line (e.g., the first and/or second purge line 124a, 124b).

Flow of the extracted gases through the sampling circuit 50 via the gas supply 102 and introduction of purge fluid into the sampling circuit 50 via the purge lines 124 can be controlled using valves or any other suitable flow control device. The flow control device(s) used to control extract gas and purge fluid flow through the sampling circuit 50 may be operator controlled and/or autonomously controlled via a controller (e.g., the computing device 120). The flow control device(s) can also be independently controlled, such that one, some, or all the purge lines 124 can be used to introduce purge fluid in conjunction with or independent from the other purge lines 124. Additionally, control of the purge fluid being introduced via one, some, or all the purge lines 124 can be performed in conjunction with or independent from controlling when the extracted gases are ceased from flowing in the sampling circuit 50 via the gas supply 102.

One or more valves 126 can be located at or proximate to a junction between each purge line 124 and the transport line 54 and/or the conduit 105. The valve(s) 126 at each junction can be positionable to either connect the gas supply 102 with a conduit (e.g., the transport line 54 or the conduit 105) of the sampling circuit 50, or to connect the purge fluid supply (e.g., outside air, cabin air, or inert gas supply) with the conduit of the sampling circuit 50 via the respective purge line 124. In the illustrated example, each valve 126 may include a switching or three-way valve that is positionable to allow flow of fluid from either the gas supply 102 or the purge fluid supply at the respective junction, while isolating the other fluid supply. Alternatively, multiple valves can be located at or in proximity to the junction to achieve a similar function. Additionally, or alternatively, in some embodiments each purge line 124 may be equipped with an isolation valve, and a main gas isolation valve can be located on the transport line 54 upstream from each of the purge lines 124 and/or adjacent to the outlet of the gas supply 102. Any number of valve arrangements or other flow control device configurations can be used to enable controlling flow of the extracted gases and the purge fluid through the sampling circuit 50 to achieve the benefits described herein of contaminant detection and analysis.

In the example of FIG. 2, three valves 126 are included and are labeled 126a-c. A first three-way valve 126a is positioned on the transport line 54 at the junction between the first purge line 126a and the transport line 54. A second three-way valve 126b is positioned on the transport line 54 at the junction between the second purge line 126b and the transport line 54. A third three-way valve 126c is positioned on the conduit 105 between the third purge line 126c and the conduit 105. Each valve 126a-c is a dedicated three-way valve that is positionable to either connect the gas supply 102 or the purge fluid supply (e.g., outside air, cabin air, or inert gas supply) to the sampling circuit 50 at the respective junction. Any number of three-way valves 126, or other suitable valves or flow control devices, can be included. In various embodiments, each junction between a purge line 126 and the sampling circuit 50 can have a dedicated valve(s) or flow control device(s).

The sampling circuit 50 (e.g., via the valves 126) can be controlled such that flow of the extracted gases and flow of the purge fluid through the sampling circuit 50 are performed at desired or specified stages. The extracted gases are suitably flowed through the sampling circuit 50 when the measurement of these gases is desired (e.g., when the gases are being obtained by and/or produced in the gas supply 102). For example, in the case of the sampling circuit 50 used with the installation 11, the extracted gases are suitably flowed through the sampling circuit 50 for measurement during a drilling operation or another standard operating stage of the installation 11 when the extracted gases/drilling mud are actively emerging from the surface 22 of the geological formation 21. The purge fluid can be flowed through the sampling circuit 50 when the measurement of the extracted gases is not needed or not available (e.g., when the gases are not being obtained by and/or produced in the gas supply 102). For example, the purge fluid can be flowed through the sampling circuit 50 for contaminant measurement when a drilling operation is not being performed at the installation 11 and/or when extracted gases/drilling mud are not actively emerging from the surface 22 (e.g., when the installation 11 is idle or when another segment of the drilling pipe is being installed). In this way, the contaminant analysis operation using the purge fluid can advantageously be performed while minimizing or eliminating any production inefficiencies or disturbances to the standard workflow.

The stages of performing the sample analysis operation in the sampling circuit 50, whereby the extracted gases are measured via the analyzer 55, and performing the contaminant analysis operation in the sampling circuit 50, whereby the purge fluid is measured via the analyzer 55, can be controlled by an operator or autonomously (e.g., via the computing device 120).

The computing device 120 may be connected in communication with one or more components of the sampling circuit 50 via connections 122, which can be wired and/or wireless connections. The computing device 120 may be a central controller for the sampling circuit 50. Additionally, or alternatively, the computing device 120 may include one or more remote or decentralized controllers that can be dedicated to one or more components of the sampling circuit 50 (e.g., the pump module 104, the analyzer 55, the valve(s) 126 etc.). In some embodiments, the computing device 120 may be connected in communication with one or more components of the installation 11 other that the sampling circuit 50. In some embodiments, the computing device 120 may be or may include a distributed network of controllers that are configured to execute and/or monitor functions of the installation 11. In some embodiments, the computing device 120 may include or may be in communication with one or more user interfaces, and can display information (e.g., alerts or information obtained by the analyzer 55) to an operator of the installation 11 and/or receive inputs from the operator of the installation 11. Articles such as “a,” “an,” and “the” used in conjunction with computing device 120 and its components encompass a single computing device 120 and multiple computing devices 120, which may be centralized, decentralized, distributed, or communicatively connected to one, some, or all components in any suitable arrangement to enable the functions described herein to be performed. Moreover, reference to “computing device,” “processor,” “memory,” or another component of the computing device in the singular encompasses one or multiple of the component unless expressly stated otherwise or the context clearly indicates otherwise.

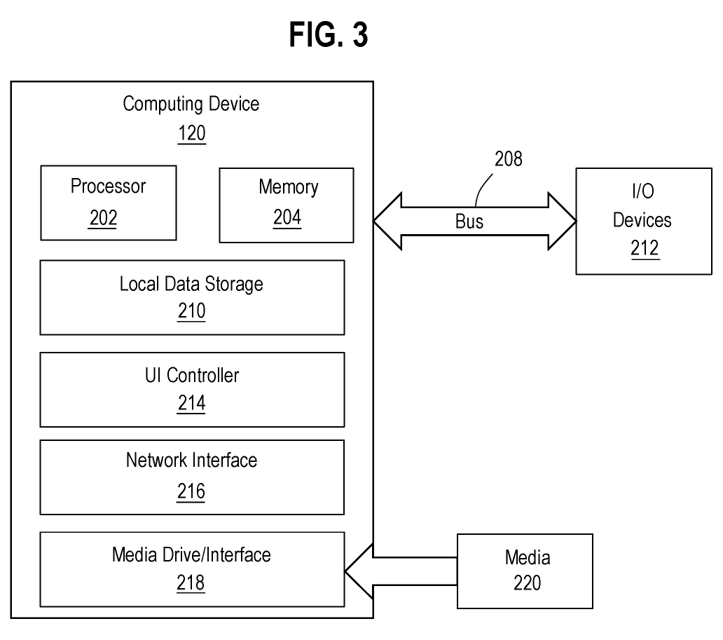

Referring now to FIG. 3, the computing device 120 includes a processor 202 and memory 204 that can store instructions executable by the processor 202 which may configure the computing device 120 to implement part, or all the functions described herein. Description herein of the computing device 120 executing a function can also be described as the processor 202 performing the function by executing the stored instruction in the memory 204. The memory 204 can also host one or more databases and can include one or more forms of volatile data storage media such as random-access memory (RAM), and/or one or more forms of nonvolatile storage media (such as read-only memory (ROM), flash memory, and so forth). The computing device 120 depicted in FIG. 3 is one example of a computing device or programmable device and is not intended to suggest any limitation as to scope of use or functionality of the computing device 120 and/or its possible architectures. For example, the computing device 120 can include one or more computing devices, programmable logic controllers (PLCs), etc. Moreover, the computing device 120 is not limited to any combination of components, such as those shown in relation to the illustrated computing device 120. The computing device 120 may include one or more of computers, such as a laptop computer, a desktop computer, a mainframe computer, etc., or any combination or accumulation thereof.

The computing device 120 can also include a bus 208 configured to allow various components and devices, such as processors 202, memory 204, and local data storage 210, among other components, to communicate with each other. The bus 208 can include one or more of any of several types of bus structures, including a memory bus or memory controller, a peripheral bus, an accelerated graphics port, and a processor or local bus using any of a variety of bus architectures. The bus 208 can also include wired and/or wireless buses. The local data storage 210 can include fixed media (e.g., RAM, ROM, a fixed hard drive, etc.) as well as removable media (e.g., a flash memory drive, a removable hard drive, optical disks, magnetic disks, and so forth).

One or more input/output (I/O) device(s) 212 may also communicate via a user interface (UI) controller 214, which may connect with I/O device(s) 212 either directly or through bus 208. In some embodiments, a network interface 216 may communicate outside of the computing device 120 via a connected network.

A media drive/interface 218 can accept removable tangible media 220, such as flash drives, optical disks, removable hard drives, software products, etc. In some embodiments, logic, computing instructions, and/or software programs comprising elements of the memory may reside on removable media 220 readable by media drive/interface 218. Various processes of the present disclosure or parts thereof can be implemented by instructions and/or software programs that are elements of the memory 204. Such instructions and/or software programs may reside on removable media 220 readable by media drive/interface 218.

In some embodiments, the input/output device(s) 212 can allow a user (such as a human operator) to enter commands and information to the device 120, and also allow information to be presented to the user and/or other components or devices. Examples of input device(s) 212 include, for example, sensors, a keyboard, a cursor control device (e.g., a mouse), a microphone, a scanner, and any other input devices known in the art. Examples of output devices include a display device (e.g., a monitor or projector), speakers, a printer, a network card, and so on.

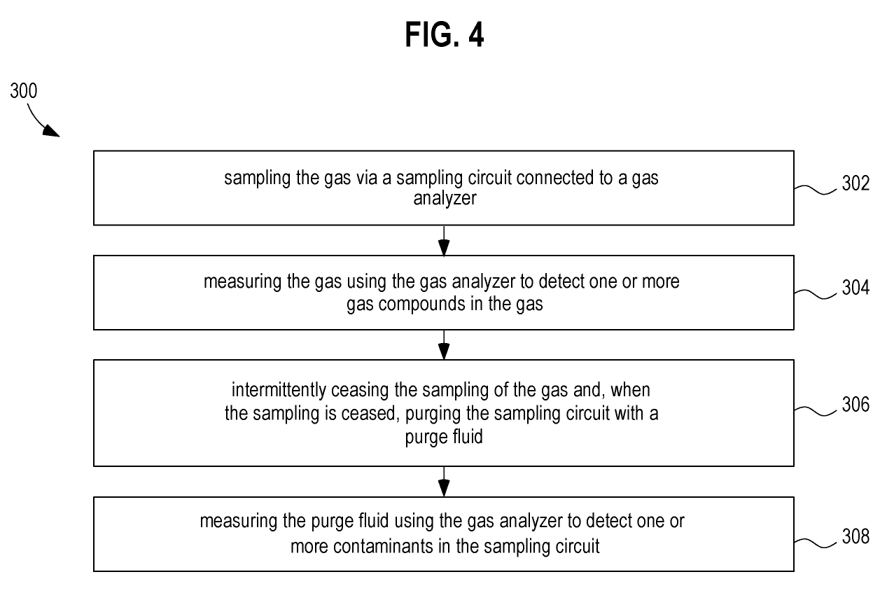

Referring now to FIG. 4, an example process flow diagram 300 according to embodiments of the present disclosure is shown, which can be implemented in the sampling circuit 50 for reliably and accurately analyzing extracted gases received from the gas supply 102. At 302, the extracted gases are sampled via the sampling circuit 50 that includes the analyzer 55, whereby the gases may be flowed through the transport line 54, the pump module 104, and the conduit 105 towards the analyzer 55. The gases may be separated at the gas separation module 116 into different gas compounds or gas fractions contained in the extracted gases, and the separated gas compounds (or fractions) may be measured at 304 using the analyzer 55. Alternatively, the extracted gases can be measured at 304 using the analyzer 55 to detect the gas compounds without first separating the extracted gases at the gas separation module 116.

The extracted gases may be measured at 304 using the detector 118 of the analyzer 55, which can obtain measurement data indicative of gas content in the sampled extracted gases. For example, the detector 118 can obtain measurement data that identifies and/or quantifies one or more gas compounds or gas fractions contained in the sampled gases. Additionally, or alternatively, the detector 118 can obtain measurement data that detects and quantifies a total gas of the sampled gases. The measurement data obtained at 304 using the analyzer 55 can be transmitted to and, optionally, stored by the computing device 120. In some embodiments, the computing device 120 may display information related to the measurement data of the sample gases, such as charts or graphs of the measurement data, to an operator (e.g., via a user interface).

The sampling and measurement operations performed at 302 and 304, respectively, can be performed over one or more cycles. Multiple cycles can be performed, which may be used to discern trends in the measurement data over the course of the sampling analysis operation. A cycle time can be specified by the operator or autonomously controlled (e.g., via the computing device 120). The cycle time may vary depending on the extracted gases and the analyte of interest. For example, in the case of short chain hydrocarbons (e.g., C₁-C₁₀ hydrocarbons), the cycle time may be between 75 seconds to 115 seconds.

Still referring to FIG. 4, at 306 the sampling of the extracted gases may be intermittently ceased for a contaminant analysis operation, where purge fluid is flowed through the sampling circuit 50. The purge fluid (e.g., air and/or inert gas) can operate to desorb any contaminants that have adsorbed in the sampling circuit 50. At 308, the purge fluid, and any contaminants that have desorbed into the purge fluid, can be measured using the analyzer 55 to detect any contaminants in the sampling circuit 50.

Flow of the purge fluid through the sampling circuit 50 can be controlled such that the purge fluid flows through all or a portion of the sampling circuit 50, as described above. For example, the purge fluid can be flowed through the sampling circuit 50 via the first purge line 126a (FIG. 2) to provide a comprehensive contaminant analysis of the sampling circuit 50. Additionally, or alternatively, the purge fluid can be flowed through the sampling circuit 50 via the second purge line 126b and/or the third purge line 126c to provide a more acute analysis of discrete components of the sampling circuit 50. In some embodiments, the purge fluid may first be flowed through the sampling circuit 50 via the third purge line 126c to for calibrating the analyzer 55 for measuring the purge fluid, followed by flowing the purge fluid via the second purge line 126b and/or third purge line 126c. The purge fluid may be measured using the detector 118, and, optionally, the purge fluid may first be separated at the gas separation module 116 prior to measurement at the detector 118.

In some embodiments, purging and measurement operations performed at 306 and 308, respectively, can be performed over one or more cycles, similar to the cycles performed for operation 302 and 304 described above. A cycle time for the operations 306 and 308, can be specified by the operator or autonomously controlled (e.g., via the computing device 120). In some embodiments, the cycle time can be adjusted in response to the detection of one or more contaminants. The cycle time may vary depending on, for example, whether any contaminants are expected and/or the nature of the contaminants. In some instances, the cycle times for the operations 306 and 308 may be longer than the cycle times for the operations 302 and 304. This may allow more comprehensive measurement data to be obtained for contaminants, which may include longer-chain hydrocarbons (e.g., hydrocarbons having a chain length of C₁₁ or longer). For example, in some embodiments, the cycle time for the purging and measurement operations performed at 306 and 308 may be greater than 115 seconds, such as between 135 seconds to 165 seconds.

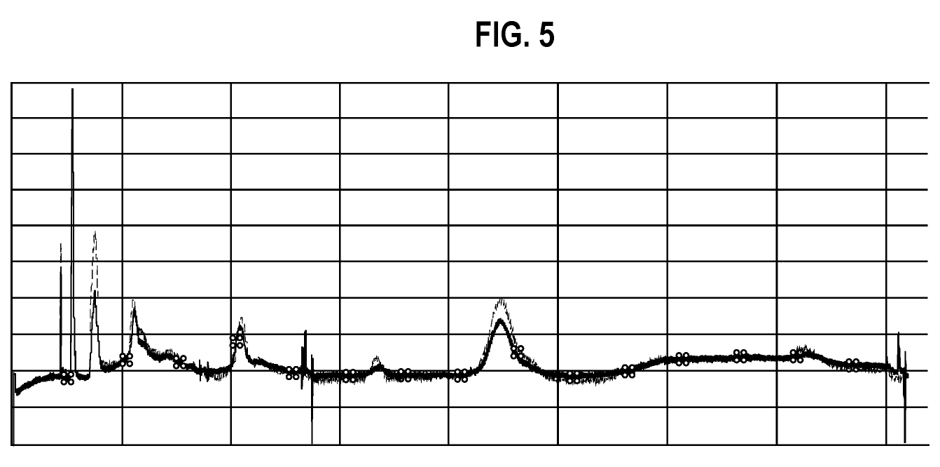

In embodiments where the operations are performed at 306 and 308 over multiple cycles, the computing device 120 may monitor the measurement data at each cycle. When contaminant is detected in the purge fluid based on the measurement data, a rate of decontamination may be monitored or discerned over the subsequent cycles. In some embodiments, the rate of decontamination can be monitored until a baseline level of contamination is detected (e.g., when the rate of decontamination reaches a steady state over multiple cycles). FIG. 5 is an example of two chromatograms that may be taken during different cycles during a contaminant analysis operation. The chromatograms are overlaid, and the drop in amplitude of the peaks indicates that decontamination is taking place.

Referring again to FIG. 4, the computing device 120, and/or the operator, may control the process flow 300 such that the operations 302 and 304 are performed when the measurement of these gases is desired, and the operations 306 and 308 are performed when the contaminant measurement of the sampling circuit 50 is desired. The stages at which the operations 302 and 304 are desired and at which the operations 306 and 308 are desired may depend, for example, on the operating state of a system used in conjunction with the sampling circuit (e.g., an operation state of the installation 11). As discussed above, for example, the operations 302 and 304 may be performed during a drilling operation or another standard operating stage of the installation 11 when the extracted gases/drilling mud are actively emerging from the surface 22 of the geological formation 21, and the operations 306 and 308 may be performed when a drilling operation is not being performed at the installation 11 and/or when extracted gases/drilling mud are not actively emerging from the surface 22 (e.g., when the installation 11 is idle or when another segment of the drilling pipe is being installed).

Like the measurement operation at 304, the measurement data obtained at 308 using the analyzer 55 can be transmitted to and, optionally, stored by the computing device 120. The computing device 120 may also display information related to the measurement data obtained during the contaminant analysis operation to the operator (e.g., via a user interface). The operator can monitor the contaminant analysis operation measurement data and, if the operator discerns that the measurement data indicates that a contaminant is detected, the operator may take appropriate remedial action.

In some embodiments, the computing device 120 may determine that the measurement data obtained at 308 is indicative of contamination in the sampling circuit 50 based on predefined thresholds and/or historical contamination analysis data that is compared to the measurement data at 308. When the presence of contaminant is detected based on measurement data obtained at 308, the computing device 120 may store an event in memory 204 associated with the measurement data that can subsequently be used for another contaminant analysis operation. In this regard, the computing device 120 can be continually refined or updated with a more accurate or comprehensive database for the detection and identification of contamination of the sampling circuit 50. The computing device 120 may store the contamination event data autonomously, or may do so in response to a user input that associates the measurement data obtained at 308 with a contamination event.

The historical contamination data may be stored in the memory 204 of the computing device 120 to obtain “fingerprint” measurement data that provides information on the contamination of the sampling circuit. The fingerprint data can be continually refined, updated, and leveraged to provide the advantages described herein, including real-time identification of contaminants and suggestion of remedial actions for specific contamination events that may vary based on different sampled gases that the sampling circuit 50 may be exposed to.

In some embodiments, the computing device 120 may alert the operator when the measurement data obtained at 308 indicates a presence of contaminant (e.g., as determined based on a comparison between the obtained measurement data and the predefined thresholds and/or historical contamination analysis data). The alert may include a visual notification or prompt to the operator via a user interface. Additionally, or alternatively, the notification can be an audible alarm or sound. In some embodiments, the alert can include a suggestion for remedial action based on the contaminant. The suggestion can be based on historical remedial actions taken in response to similar contamination analysis data, which may also be stored by the computing device 120 in the memory 204. The suggestion may include, for example, switching to another pump module 104 or changing components in the sampling circuit 50. The suggestion can be further refined based on more acute contaminant analysis, which can be performed as described above to further pinpoint discrete locations in the sampling circuit 50 that are contaminated.

In some embodiments, the computing device 120 may autonomously take a remedial action when the measurement data obtained at 308 indicates a presence of contaminant (e.g., as determined based on a comparison between the obtained measurement data and predefined thresholds and/or historical contamination analysis data). For example, the computing device 120 may adjust a measurement setting of the analyzer 55 to compensate for the presence of the contamination in the sampling circuit 50. The adjusted measurement setting could be a correction to the measurement data obtained at 304 during a sample analysis operation. The correction may include omitting from the measurement data obtained at 304 any data related to the contaminant, and/or re-calibrating the analyzer 55 to account for the contaminant in its baseline. Additionally, or alternatively, the computing device 120 may adjust a measurement setting during subsequent cycles of the contaminant analysis operation, such as by adjusting a cycle time for the operations 306 and 308.

The embodiments described above in accordance with this disclosure are therefore advantageous in that they allow for the real-time detection of contaminants in a sampling circuit 50, and enable the appropriate course of action to be taken in response to a contamination event for obtaining more accurate and reliable measurement data of gases using the sampling circuit 50. Moreover, the embodiments described herein enable a comprehensive and accurate database of contaminant information to be built, which can be leveraged to identify specific contaminants that are not otherwise identifiable or discernible by operators in the field. Furthermore, the contaminant analysis can be performed in an efficient and cost-effective manner, while reducing or avoiding any production downtime or process disruptions during the contaminant analysis.

Various processes or parts of the workflow of the present disclosure may be described herein in the general context of software or program modules, or the techniques and modules may be implemented in pure computing hardware. Software generally includes routines, programs, objects, components, data structures, and so forth that perform particular tasks or implement particular abstract data types. An implementation of these modules and techniques may be stored on or transmitted across some form of tangible computer-readable media. Computer-readable media can be any available data storage medium or media that is tangible and can be accessed by a computing device. Computer readable media may thus comprise computer storage media. “Computer storage media” designates tangible media, and includes volatile and non-volatile, removable and non-removable tangible media implemented for storage of information such as computer readable instructions, data structures, program modules, or other data. Computer storage media include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible medium which can be used to store the desired information, and which can be accessed by a computer.

In embodiments, any one or any portion or all of the steps or operations of the workflow as described above can be performed by a processor. The term “processor” should not be construed to limit the embodiments disclosed herein to any particular device type or system. The processor may include a computer system. The computer system may also include a computer processor (e.g., a microprocessor, microcontroller, digital signal processor, or general-purpose computer) for executing any of the methods and processes described above.

The computer system may further include a memory such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device.

Some of the methods and processes described above, can be implemented as computer program logic for use with the computer processor. The computer program logic may be embodied in various forms, including a source code form or a computer executable form. Source code may include a series of computer program instructions in a variety of programming languages (e.g., an object code, an assembly language, or a high-level language such as C, C++, or JAVA). Such computer instructions can be stored in a non-transitory computer readable medium (e.g., memory) and executed by the computer processor. The computer instructions may be distributed in any form as a removable storage medium with accompanying printed or electronic documentation (e.g., shrink wrapped software), preloaded with a computer system (e.g., on system ROM or fixed disk), or distributed from a server or electronic bulletin board over a communication system (e.g., the Internet or World Wide Web).

Alternatively or additionally, the processor may include discrete electronic components coupled to a printed circuit board, integrated circuitry (e.g., Application Specific Integrated Circuits (ASIC)), and/or programmable logic devices (e.g., a Field Programmable Gate Arrays (FPGA)). Any of the methods and processes described above can be implemented using such logic devices.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the present disclosure, additionally, they do not exclude impurities and variances normally associated with the elements and materials used.

Numerical ranges used herein include the numbers recited in the range. For example, the numerical range “from 1 wt % to 10 wt %” includes 1 wt % and 10 wt % within the recited range.

For the sake of brevity, only some ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All numerical values within the detailed description herein are modified by “about” the indicated value, and take into account experimental error and variations that would be expected by a person having ordinary skill in the art.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, references to “one embodiment” or “an embodiment” of the present disclosure are not intended to be interpreted as excluding the existence of additional embodiments that also incorporate the recited features. The terms of “upstream” and “downstream” are understood relatively to the normal direction of circulation of a fluid in a conduit.

All documents described herein are incorporated by reference herein, including any priority documents and or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

The specific embodiments described herein have been illustrated by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function)…” or “step for (perform)ing (a function)…”, it is intended that such elements are to be interpreted under 35 U.S.C. 112(f). However, for any claims containing elements designated in any other manner, it is intended that such elements are not to be interpreted under 35 U.S.C. 112(f).

### Embodiments

Implementation examples are described in the following numbered clauses:

E1. A gas analysis system, the system comprising:

(a) a sampling circuit comprising:

a conduit connectable to a gas supply for receiving a gas sample during a sample analysis operation and to a purge fluid supply for receiving a purge fluid during a contaminant analysis operation; and

at least one valve positionable to enable connection between the conduit and the gas supply during the sample analysis operation, and to enable connection between the conduit and the purge fluid supply during the contaminant analysis operation; and

(b) a gas analyzer connected to the sampling circuit, the gas analyzer being operable to obtain measurement data indicative of gas content in the gas sample during the sample analysis operation and measurement data indicative of gas content in the purge fluid during the contaminant analysis operation.

E2. The gas analysis system of embodiment E1, wherein the sampling circuit further comprises a pump module that moves the gas sample and the purge fluid through the conduit towards the gas analyzer.

E3. The gas analysis system of embodiment E2, wherein the at least one valve is located upstream from the pump module.

E4. The gas analysis system of embodiment E2, wherein the at least one valve is located downstream from the pump module.

E5. The gas analysis system of any one of embodiments E1 to E4, wherein the gas supply is part of a well site installation and the gas sample includes gas extracted from a drilling fluid exiting a wellbore.

E6. The gas analysis system of embodiment E5, further comprising a controller configured to control positioning of the at least one valve based on an operating state of the well site installation.

E7. The gas analysis system of embodiment E6, wherein the controller is configured to position the at least one valve to connect the conduit and the gas supply when a drilling operation is being performed at the well site installation, and to position the at least one valve to connect the conduit and the purge fluid supply when an operation other than the drilling operation is being performed at the well site installation.

E8. The gas analysis system of any one of embodiments E1 to E7, wherein the gas analyzer includes a gas chromatograph separating gas compounds in the gas sample and the purge fluid and a detector measuring the extracted gas compounds.

E9. The gas analysis system of embodiment E8, wherein a cycle time of the gas analyzer is longer during the contaminant analysis operation than during the sample analysis operation.

E10. The gas analysis system of any one of the embodiments E1 to E9, wherein the gas analyzer includes a computing device having memory storing the measurement data of the gas analyzer.

E11. The gas analysis system of embodiment E10, wherein the computing device alerts an operator when the measurement data of the purge fluid indicates a presence of contaminant.

E12. The gas analysis system of embodiment E10 or embodiment E11, wherein the computing device stores an event associated with the measurement data in the memory when the measurement data of the purge fluid indicates a presence of contaminant.

E13. The gas analysis system of any one of embodiments E10 to E12, wherein the computing device adjusts a measurement setting of the gas analyzer when the measurement data of the purge fluid indicates a presence of contaminant.

E14. The gas analysis system of embodiment E13, wherein the measurement setting includes a cycle time of the gas analyzer during the contaminant analysis operation.

E15. The gas analysis system of embodiment E13 or embodiment E14, wherein the measurement setting includes a correction of the measurement data obtained during the sample analysis operation based on the measurement data obtained during the contaminant analysis operation to compensate for the contaminant in the purge fluid.

E16. A well site installation for analyzing drilling fluid exiting a wellbore, the installation comprising:

(a) a pipe drawing the drilling fluid from the wellbore;

(b) a sampling circuit in fluid communication with the pipe, the sampling circuit comprising:

an extractor operable to separate gas contained in the drilling fluid;

a conduit connectable to the extractor for receiving a sample of the gas during a sample analysis operation and to a purge fluid supply for receiving a purge fluid during a contaminant analysis operation; and

at least one valve positionable to enable connection between the conduit and the extractor during the sample analysis operation, and to enable connection between the conduit and the purge fluid supply during the contaminant analysis operation; and

(c) a gas analyzer connected to the sampling circuit, the gas analyzer being operable to obtain measurement data indicative of gas content in the sample of the gas during the sample analysis operation and measurement data indicative of gas content in the purge fluid during the contaminant analysis operation.

E17. The well site installation of embodiment E16, further comprising a controller configured to control positioning of the at least one valve based on an operating state of the well site installation.

E18. The well site installation of embodiment E17, wherein the controller is configured to position the at least one valve to connect the conduit and the extractor for the sample analysis operation when a drilling operation is being performed at the well site installation, and to position the at least one valve to connect the conduit and the purge fluid supply for the contaminant analysis operation when the well site installation when an operation other than the drilling operation is being performed at the well site installation.

E19. The well site installation of any one of embodiments E16 to E18, wherein the gas analyzer includes a computing device having memory storing the measurement data of the gas analyzer.

E20. The well site installation of embodiment E19, wherein the computing device alerts an operator when the measurement data of the purge fluid indicates a presence of contaminant.

E21. The well site installation of embodiment E19 or embodiment E20, wherein the computing device stores an event in the memory when the measurement data of the purge fluid indicates a presence of contaminant.

E22. The well site installation of any one of embodiments E19 to E21, wherein the computing device adjusts a measurement setting of the gas analyzer when the measurement data of the purge fluid indicates a contaminant in the purge fluid.

E23. The well site installation of embodiment E22, wherein the measurement setting includes a cycle time of the gas analyzer during the contaminant analysis operation.

E24. The well site installation of embodiment E22 or E23, wherein the measurement setting includes a correction of the measurement data obtained during the sample analysis operation based on the measurement data obtained during the contaminant analysis operation to compensate for the contaminant in the purge fluid.

E25. A gas analysis system comprising:

(a) a sampling circuit connectable to a gas supply for receiving a gas sample during a sample analysis operation;

(b) a gas analyzer connected to the sampling circuit, the gas analyzer being operable to obtain measurement data indicative of gas content in the gas sample during the sample analysis operation and to obtain measurement data indicative of contaminant in the sampling circuit when the sample analysis operation is not being performed; and

(c) a computing device electronically connected to the gas analyzer and including a processor and a memory storing instructions that, when executed by the processor, cause the processor to:

detect the contaminant in the sampling circuit based on the measurement data obtained by the gas analyzer when the sample analysis operation is not being performed; and

in response to the detected contaminant, adjust a measurement setting of the gas analyzer.

E26. The gas analysis system of embodiment E25, wherein the measurement setting includes a cycle time of the gas analyzer when obtaining the measurement data indicative of the contaminant in the sampling circuit.

E27. The gas analysis system of embodiment E25 or embodiment E26, wherein the measurement setting includes a correction of the measurement data obtained during the sample analysis operation to compensate for the contaminant detected in the sampling circuit.

E28. The gas analysis system of any one of embodiments E25 to E27, wherein the processor alerts an operator when the contaminant is detected in the sampling circuit.

E29. The gas analysis system of embodiment E28, further comprising a user interface electronically connected to the processor, wherein the processor alerts the operator by causing display of an alert notification on the user interface.

E30. The gas analysis system of any one of embodiments E25 to E29, wherein the processor stores, in the memory, the measurement data obtained by the gas analyzer when the contaminant is detected in the sampling circuit, and associates the stored measurement data with a contamination event.

E31. The gas analysis system of embodiment E30, wherein the processor compares the measurement data obtained by the gas analyzer when the contaminant is detected in the sampling circuit with historical measurement data stored in the memory associated with historical contamination events.

E32. The gas analysis system of embodiment E31, wherein, when the comparison indicates a match between the obtained measurement data and the historical measurement data, the processor performs at least one of: notify an operator of the match to prompt a remedial action; and identify the contaminant in the sampling circuit using the historical measurement data.

E33. The gas analysis system of any one of embodiments E25 to E32, wherein: the gas analyzer is operable to obtain the measurement data indicative of the contaminant in the sampling circuit over multiple measurement cycles, and when the processor detects the contaminant in the sampling circuit during one of the measurement cycles, the processor monitors a rate of decontamination of the sampling circuit during one or more subsequent measurement cycles.

E34. The gas analysis system of any one of embodiments E25 to E33, wherein the gas supply is part of a well site installation and the gas sample includes gas extracted from a drilling fluid exiting a wellbore.

E35. The gas analysis system of embodiment E34, wherein the processor controls connection between the conduit and the gas supply based on an operating state of the well site installation.

E36. The gas analysis system of embodiment E35, wherein the processor controls connection between the conduit and the gas supply when a drilling operation is being performed at the well site installation, and controls connection between the conduit and a purge fluid supply when an operation other than the drilling operation is being performed at the well site installation.

E37. A well site installation for analyzing drilling fluid exiting a wellbore, the installation comprising:

(a) a pipe drawing the drilling fluid from the wellbore;

(b) a sampling circuit in fluid communication with the pipe, the sampling circuit operable to separate gas contained in the drilling fluid and receive a sample of the gas during a sample analysis operation;

(c) a gas analyzer connected to the sampling circuit, the gas analyzer being operable to obtain measurement data indicative of gas content in the gas sample during the sample analysis operation and to obtain measurement data indicative of contaminant in the sampling circuit when the sample analysis operation is not being performed; and

(d) a computing device electronically connected to the gas analyzer and including a processor and a memory storing instructions that, when executed by the processor, cause the processor to:

detect the contaminant in the sampling circuit based on the measurement data obtained by the gas analyzer when the sample analysis operation is not being performed; and

in response to the detected contaminant, adjust a measurement setting of the gas analyzer.

E38. The well site installation of embodiment E37, wherein the measurement setting includes a cycle time of the gas analyzer when obtaining the measurement data indicative of the contaminant in the sampling circuit.

E39. The well site installation of embodiment E37 or E38, wherein the measurement setting includes a correction of the measurement data obtained during the sample analysis operation to compensate for the contaminant detected in the sampling circuit.

E40. The well site installation of any one of embodiments E37 to E39, wherein the processor alerts an operator when the contaminant is detected in the sampling circuit.

E41. The well site installation of embodiment E40, further comprising a user interface electronically connected to the processor, wherein the processor alerts the operator by causing display of an alert notification on the user interface.

E42. The well site installation of any one of embodiments E37 to E41, wherein the processor stores, in the memory, the measurement data obtained by the gas analyzer when the contaminant is detected in the sampling circuit, and associates the stored measurement data with a contamination event.

E43. The well site installation of embodiment E42, wherein the processor compares the measurement data obtained by the gas analyzer when the contaminant is detected in the sampling circuit with historical measurement data stored in the memory associated with historical contamination events.

E44. The well site installation of embodiment E43, wherein, when the comparison indicates a match between the obtained measurement data and the historical measurement data, the processor performs at least one of: notify an operator of the match to prompt a remedial action; and identify the contaminant in the sampling circuit using the historical measurement data.

E45. The well site installation of any one of embodiments E37 to E44, wherein: the gas analyzer is operable to obtain the measurement data indicative of the contaminant in the sampling circuit over multiple measurement cycles, and when the processor detects the contaminant in the sampling circuit during one of the measurement cycles, the processor monitors a rate of decontamination of the sampling circuit during one or more subsequent measurement cycles.

E46. A method of analyzing gas contained in a working fluid, the method comprising: (a) sampling the gas via a sampling circuit connected to a gas analyzer; (b) measuring the gas using the gas analyzer to detect one or more gas compounds in the gas; (c) intermittently ceasing the sampling of the gas and, when the sampling is ceased, purging the sampling circuit with a purge fluid; and (d) measuring the purge fluid using the gas analyzer to detect one or more contaminants in the sampling circuit.

E47. The method of embodiment E46, further comprising extracting the gas from the working fluid prior to sampling the gas.

E48. The method of embodiment E46 or embodiment E47, further comprising, in response to detecting the one or more contaminants, adjusting a measurement setting of the gas analyzer.

E49. The method of any one of embodiments E46 to E48, further comprising, in response to detecting the one or more contaminants, identifying the one or more contaminants using historical measurement data of the gas analyzer associated with contamination of the sampling circuit.

E50. The method of any one of embodiments E46 to E49, further comprising, in response to detecting the one or more contaminants, alerting an operator of contamination of the sampling circuit.

E51. The method of any one of embodiments E46 to E50, wherein the working fluid is a drilling fluid exiting a wellbore and the gas includes one or more hydrocarbons.

E52. The method of embodiment E51, wherein the one or more contaminants include hydrocarbons adsorbed in the sampling circuit.

E53. The method of any one of embodiments E46 to E52, wherein the purge fluid includes air and/or an inert gas.

While the present disclosure has been described with respect to a number of embodiments and examples, those skilled in the art, having benefit of this disclosure, will appreciate that other embodiments can be devised which do not depart from the scope and spirit of the present disclosure.

What is claimed is:

1. A gas analysis system, the system comprising:
(a) a sampling circuit comprising:
a conduit connectable to a gas supply for receiving a gas sample during a sample analysis operation and to a purge fluid supply for receiving a purge fluid during a contaminant analysis operation; and
at least one valve positionable to enable connection between the conduit and the gas supply during the sample analysis operation, and to enable connection between the conduit and the purge fluid supply during the contaminant analysis operation; and
(b) a gas analyzer connected to the sampling circuit, the gas analyzer being operable to obtain measurement data indicative of gas content in the gas sample during the sample analysis operation and measurement data indicative of gas content in the purge fluid during the contaminant analysis operation.

2. The gas analysis system of claim 1, wherein the sampling circuit further comprises a pump module that moves the gas sample and the purge fluid through the conduit towards the gas analyzer.

3. The gas analysis system of claim 2, wherein the at least one valve is located upstream from the pump module.

4. The gas analysis system of claim 2, wherein the at least one valve is located downstream from the pump module.

5. The gas analysis system of claim 1, wherein the gas supply is part of a well site installation and the gas sample includes gas extracted from a drilling fluid exiting a wellbore.

6. The gas analysis system of claim 5, further comprising a controller configured to control positioning of the at least one valve based on an operating state of the well site installation.

7. The gas analysis system of claim 6, wherein the controller is configured to position the at least one valve to connect the conduit and the gas supply when a drilling operation is being performed at the well site installation, and to position the at least one valve to connect the conduit and the purge fluid supply when an operation other than the drilling operation is being performed at the well site installation.

8. The gas analysis system of claim 1, wherein the gas analyzer includes a gas chromatograph separating gas compounds in the gas sample and the purge fluid and a detector measuring the extracted gas compounds.

9. The gas analysis system of claim 8, wherein a cycle time of the gas analyzer is longer during the contaminant analysis operation than during the sample analysis operation.

10. The gas analysis system of claim 1, wherein the gas analyzer includes a computing device having memory storing the measurement data of the gas analyzer;
wherein the computing device stores an event associated with the measurement data in the memory when the measurement data of the purge fluid indicates a presence of contaminant; and
wherein the computing device alerts an operator when the measurement data of the purge fluid indicates a presence of contaminant.

11. The gas analysis system of claim 10, wherein the computing device adjusts a measurement setting of the gas analyzer when the measurement data of the purge fluid indicates a presence of contaminant.

12. The gas analysis system of claim 11, wherein the measurement setting includes a correction of the measurement data obtained during the sample analysis operation based on the measurement data obtained during the contaminant analysis operation to compensate for the contaminant in the purge fluid.

13. A well site installation for analyzing drilling fluid exiting a wellbore, the installation comprising:
(a) a pipe drawing the drilling fluid from the wellbore;
(b) a sampling circuit in fluid communication with the pipe, the sampling circuit comprising:
an extractor operable to separate gas contained in the drilling fluid;
a conduit connectable to the extractor for receiving a sample of the gas during a sample analysis operation and to a purge fluid supply for receiving a purge fluid during a contaminant analysis operation; and
at least one valve positionable to enable connection between the conduit and the extractor during the sample analysis operation, and to enable connection between the conduit and the purge fluid supply during the contaminant analysis operation; and
(c) a gas analyzer connected to the sampling circuit, the gas analyzer being operable to obtain measurement data indicative of gas content in the sample of the gas during the sample analysis operation and measurement data indicative of gas content in the purge fluid during the contaminant analysis operation.

14. The well site installation of claim 13, further comprising a controller configured to control positioning of the at least one valve based on an operating state of the well site installation.

15. The well site installation of claim 14, wherein the controller is configured to position the at least one valve to connect the conduit and the extractor for the sample analysis operation when a drilling operation is being performed at the well site installation, and to position the at least one valve to connect the conduit and the purge fluid supply for the contaminant analysis operation when an operation other than the drilling operation is being performed at the well site installation.

16. The well site installation of claim 13, wherein the gas analyzer includes a computing device having memory storing the measurement data of the gas analyzer;
wherein the computing device stores an event in the memory when the measurement data of the purge fluid indicates a presence of contaminant;
and wherein the computing device alerts an operator when the measurement data of the purge fluid indicates a presence of contaminant.

17. The well site installation of claim 16, wherein the computing device adjusts a measurement setting of the gas analyzer when the measurement data of the purge fluid indicates a contaminant in the purge fluid.

18. The well site installation of claim 17, wherein the measurement setting includes a cycle time of the gas analyzer during the contaminant analysis operation.

19. The well site installation of claim 17, wherein the measurement setting includes a correction of the measurement data obtained during the sample analysis operation based on the measurement data obtained during the contaminant analysis operation to compensate for the contaminant in the purge fluid.

20. A method of analyzing gas contained in a working fluid, the method comprising:
(a) sampling the gas via a sampling circuit connected to a gas analyzer;
(b) measuring the gas using the gas analyzer to detect one or more gas compounds in the gas;
(c) intermittently ceasing the sampling of the gas and, when the sampling is ceased, purging the sampling circuit with a purge fluid; and
(d) measuring the purge fluid using the gas analyzer to detect one or more contaminants in the sampling circuit.
